(19) United States
(12) Patent Application Publication
(10) Pub. No.: US 2014/0302515 A1
(43) Pub. Date: Oct. 9, 2014

(54) BIOMARKERS FOR BREAST CANCER PATIENTS

(71) Applicants: Clayton Yates, Auburn, AL (US); Timothy Turner, Auburn, AL (US); Jacqueline Jones-Triche, Tuskegee, AL (US)

(72) Inventors: Clayton Yates, Auburn, AL (US); Timothy Turner, Auburn, AL (US); Jacqueline Jones-Triche, Tuskegee, AL (US)

(73) Assignee: TUSKEGEE UNIVERSITY, Tuskegee, AL (US)

(21) Appl. No.: 13/571,106

(22) Filed: Aug. 9, 2012

Related U.S. Application Data
(63) Continuation of application No. 13/313,970, filed on Dec. 7, 2011, now abandoned.
(60) Provisional application No. 61/420,618, filed on Dec. 7, 2010.

(51) Int. Cl. G01N 33/574; A61N 5/10

(52) U.S. Cl. CPC G01N 33/57415; G01N 33/57434; A61N 5/10; G01N 2033/57403
USPC 435/7.1

(57) ABSTRACT

Kaiso, a transcriptional regulator with bimodal DNA-binding specificity, is over-expressed in breast cancer cells, and its nuclear localization is correlated with malignant and lymph node metastasis. Depleted expression of Kaiso in those cells results in a significant decrease in basal and EGF-induced cell migration, and this is associated with increased E-cadherin expression. The data reported and described herein provide significant evidence that Kaiso is involved in breast cancer invasion and metastasis. Kaiso nuclear localization is a biomarker that is associated with invasive and metastatic infiltrating ductal breast cancer that may be used to monitor, detect, and/or track the progress of disease in a patient.

### CROSS-REFERENCE TO RELATED APPLICATIONS

This application is a continuation of U.S. patent application Ser. No. 13/313,970 (filed Dec. 7, 2011), which claims the benefit of priority to prior co-pending and co-owned U.S. provisional patent application No. 61/420,618, filed Dec. 7, 2010, the entirety of which is hereby incorporated by reference.

### STATEMENT REGARDING FEDERALLY SPONSORED RESEARCH OR DEVELOPMENT

This invention was made with government support under PC07397 awarded by the Department of Defense, and award number 1 U54-CA118623-01 awarded by the NIH National Cancer Institute. The government has certain rights in the invention.

### FIELD OF THE INVENTION

The present disclosure relates to biomarkers that are useful for the detection of cancer and metastasis thereof. The invention further relates to biomarkers and methods of using biomarkers for the early detection of prostate and breast cancer and/or metastasis thereof.

### BACKGROUND OF THE INVENTION

Prostate and Breast cancer are the most commonly diagnosed malignancy in men and women, respectively, and African Americans in particular experience much higher rates of diagnosis and typically are initially diagnosed with later stages of the disease. Studies approximate that 50% have clinically advanced disease at the time of diagnosis. Transformed but not yet malignant epithelial cells in the primary tumor undergo a number of genetic and epigenetic changes that promote neoplastic outgrowth.

Additionally, the growth of human breast tumors is dependent on several factors including their hormone or estrogen-responsiveness and expression of the estrogen receptor and other gene products. Many estrogen-responsive tumors are initially treated with anti-estrogens; however, these tumors often progress to hormone-independence and become resistant to endocrine therapies. Hormone-independent tumors are more aggressive, and the development of clinical protocols for identification of when cancers are prone to transition into hormone-independence would assist clinicians in identifying the appropriate therapy protocol to undertake.

Epigenetic changes usually result from changes in cytosine (CpG) methylation or DNA-associated proteins. Specific recognition of methylated DNA is mediated either via two classes of proteins: methyl-CpG binding domain (MBD) or C₂H₂ zinc fingers such as Kaiso. Methyl-CpG binding proteins specifically recognize 5-methylcytosine and can act as intermediates between the transcriptional machinery and methylated DNA. Of the methyl-binding proteins, Kaiso has 10-fold higher affinity and represses transcription at least in part by recruiting the N-CoR corepressor.

Kaiso is a member of the BTB/POZ (Broad Complex, Tramtrak, Bric-a-brac/Pox virus and Zinc finder) zinc finger superfamily, contains a carboxyl-terminal region with three zinc finger motifs of the C₂H₂ type and recognizes clusters of methylated CpG dinucleotides as well as sequence-specific Kaiso binding site (“KBS”). Several other members of the BTB/POZ family, such as HIC and BCL6, have been reported to have a role in development and cancer as well.

In advanced stages of cancer, which are characterized by increased aggressiveness and/or invasiveness, the expression of many tumor-suppressor genes are downregulated. Increased aggressiveness and invasiveness allow the cells of a tumor mass to invade local tissue and metastasize. Kaiso is a bi-modal transcription factor that can interact with DNA through either a DNA consensus sequence or methylated CpG nucleotides, thus regulating gene expression. A clinical role for Kaiso expression in certain cancers has been identified, but its role in breast cancer and the various stages thereof has not yet been identified.

Thus, suitable biomarkers for the detection of patients that have or may develop prostate and breast cancers is desirable.

Thus, there remains a need in the art for improved treatments for breast cancer that are highly effective in causing disease remission and in preventing progression of the disease to more advanced and aggressive stages while still exhibiting low toxicity.

### SUMMARY OF THE INVENTION

In light of the above needs, it is an object of the present invention to identify improved methods for identifying, characterizing, and monitoring the state of breast cancer in patients.

The present invention achieves the above-described and other objects via the discovery that Kaiso is over-expressed in prostate and breast cancer cells, and its nuclear localization is correlated with malignant and lymph node metastasis. Further, depleted expression of Kaiso in those cells results in a significant decrease in basal and EGF-induced cell migration, and this is associated with increased E-cadherin expression. The data reported and described herein provide significant evidence that Kaiso is involved in prostate and breast cancer invasion and metastasis. Moreover, the embodiments relate to the finding that nuclear Kaiso localization is associated with more aggressive cell lines and regulated through EGFR signaling in prostate cancer. Kaiso nuclear localization is a biomarker that is associated with invasive and metastatic infiltrating prostate and ductal breast cancer that may be used to monitor, detect, and/or track the progress of disease in a patient.

As such, the biomarker according to the present invention will provide clinical support to further aid in prognosis of prostate and breast cancer. Furthermore, according to the invention nuclear Kaiso expression may be used as an indicator that local and/or metastasis has occurred in patients.

The experimental data reported herein further suggests that modification or depletion of the Kaiso gene would serve as a reasonable therapeutic approach to limit cancer metastasis for prostate and breast cancer. Further, given the significant number of metastasis related genes that Kaiso has been shown to control, detection of nuclear Kaiso can significantly aid in determining the appropriate mode of therapy for cancer patients.

To practice methods relating to the early detection of cancer, biomarker expression levels of sample biopsies from persons believed to be at risk for developing cancer are compared to the expression levels of control biopsies (normal biopsy that does not have cancer) and advanced stage cancer biopsies. If sample biomarker expressions levels are higher than normal control biomarker expression levels, cancer has been detected. Further, if sample biomarker expressions levels are higher than normal control biomarker expression levels, but less than cancer biopsies, then cancer has been detected early.

In this regard, one embodiment of the invention relates to a method for the detection of prostate or breast cancer in a subject. This method includes obtaining a sample biopsy of prostate or breast tissue from a subject, determining an expression level of Kaiso in the sample biopsy; and comparing the expression level of Kaiso in said sample biopsy with an average expression level of Kaiso for a healthy control biopsy and an average expression level of Kaiso for a cancerous control biopsy. In this method, the cancerous control biopsy is of a known prostate or breast cancer stage, and a determination that the level of expression of Kaiso in the sample biopsy is closer to the average level of expression of Kaiso in the cancerous control biopsy than the average level of expression of Kaiso in the healthy control biopsy leads to a diagnosis of the subject as having prostate or breast cancer, and a determination that the level of expression of Kaiso in the sample biopsy is greater than the average level of expression of Kaiso in said cancerous control biopsy leads to a diagnosis of the subject as having prostate or breast cancer of this stage.

In another embodiment, the invention relates to a method for monitoring the stage of prostate or breast cancer in a subject. The method includes measuring the expression level of Kaiso in prostate or breast tissue of the subject, comparing the measured expression level of Kaiso with average expression levels of Kaiso for at least two prostate or breast cancer stages, and identifying the subject as having cancer of a particular stage depending upon where said measured expression level falls relative to said average expression levels.

In yet another embodiment, the invention relates to a method for detecting potential metastasis of prostate or breast cancer in a subject. This method includes measuring the expression level of Kaiso in prostate or breast tissue of the subject, comparing the measured expression level of Kaiso with average expression levels of Kaiso for at least two prostate or breast cancer stages. At least one of the stages is characterized by metastasis and at least another one of the stages is characterized by no metastasis. The method further includes identifying the subject as having increased risk for metastasis if the measured expression level falls relatively closer to the average expression level for the stages being characterized by metastasis.

Further, other embodiments of the invention relate to methods for treating a subject diagnosed with prostate or breast cancer. These treatment methods comprise providing a therapy appropriate for a stage of prostate or breast cancer as identified according to any of the detection methods according to the present invention.

In accordance with another embodiment, the invention relates to methods of using biomarkers for the early detection of cancer and/or for the monitoring of cancer progression in a patient.

In any of the methods of the invention, a current stage of prostate or breast cancer can be determined for the subject based upon where a measured level of Kaiso in the sample biopsy (or other test sample) falls in comparison to said average levels. Such stages can comprise or be otherwise indicative of metastasis, impending metastasis, hormone independent prostate or breast cancer, impending hormone independent prostate or breast cancer, aggressive prostate or breast cancer, impending aggressive prostate or breast cancer, and/or infiltrating duct prostate or breast cancer.

Optionally, the measured levels and/or prostate or breast tissue samples can be taken from a pathology sample from a cancerous tumor or other lump of tissue surgically removed from said subject, by biopsy needle, or other suitable methods. Further, any method of the present invention can advantageously be employed in conjunction with chemotherapy and/or radiotherapy to appropriately match therapies to the types and/or stages of prostate or breast cancer possessed by the subject.

Further, different average control levels can be used based upon racial or ethnic profiles of said subject.

The various embodiments of the invention having thus been generally described, several illustrative embodiments will hereafter be discussed with particular reference to several attached drawings and in view of various experimental examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A through FIG. 1C are tables reporting the experimental results of Kaiso expression levels obtained from breast tissue samples taken from various human patients.

FIG. 2 is a scatter plot reporting experimental data for all of the human patient samples analyzed for the overall, nuclear, cytoplasmic, and membranous Kaiso expression levels.

FIG. 3 is a graph comparing the Kaiso expression levels for Caucasian and African American patients obtained in various experiments described further herein.

FIG. 4 comprises black and white photographs of representative gels obtained in experiments described herein probing for mRNA Kaiso levels and protein for the three different breast cancer cell lines.

FIG. 5 is a grid of twelve color immunofluorescence photographs for the three breast cancer cell lines.

FIG. 6 is a bar graph showing the relative threshold intensity identified for each of three breast cancer cell lines.

FIG. 7 is bar graph showing the relative levels of Kaiso expression obtained in MDA-MB-231 cells using two different siRNA Kaiso constructs.

FIG. 8 comprises an array of six black and white photographs of representative cell cultures for the cell migration assay for MDA-MB-231 cells at zero and 24 hours.

FIG. 9 is bar graph reporting Applicants' experimental results for the relative migration of MDA-MB-468 and MDA-MB-231 cell lines.

FIG. 10 is a bar graph reporting Applicants' experimental results for the number of invading cells for the MDA-MB-231 cell line.

### DETAILED DESCRIPTION OF THE PREFERRED EMBODIMENTS

Unless otherwise set forth herein, cell cultures were grown in high glucose Dulbecco's modified Eagles medium (“DMEM”) supplemented with fetal bovine serum (FBS; 10%), penicillin-streptomycin, sodium pyruvate, non-essential amino acids, and L-glutamine at 37° C., 90% humidity, 5% CO₂ and 95% air.

Unless otherwise set forth herein, immunofluorescence studies comprised plating cells on a 4-chamber slide with 20000 cells. After treatment, they were then washed with PBS, fixed for 30 minutes with cold methanol at room temperature, incubated at room temperature with Lysis Buffer containing 0.1 M Tris, 0.15M NaCl, 0.01 M EGTA, 1% Triton and 1 mM PMSF, blocked for 30 mins in 5% BSA dissolved in PBS, treated with primary antibody (1:100) overnight in the cold room. Cells were stained for p120^{ctn} and Kaiso.

Unless otherwise set forth herein, siRNA transfections were accomplished utilizing the following general procedures. Cells were incubated until 60-80% confluent. Prepared the following solutions: A: 6 μl of siRNA (Santa Cruz Biotech) into 100 μL of Opti Medium B: 6 μL of Lipofectamine 2000 Reagent (Invitrogen) into 100 μl of Opti Medium. Added solution A to B and let incubate at room temperature for 15-45 minutes. During that time washed cells twice with 2 ml of Opti Medium. Once incubation was done added 800 μl to make a total of 1 ml and then treated cells for 24 hrs. Once treated with 24 hours let cells recover in normal growth conditions.

In the various experiments described herein, unless otherwise described monoclonal antibodies to p120^{ctn} and Kasio where obtained commercially, such as from BD Transduction Laboratories. Immunofluorsecent staining for Kaiso was done with Kaiso 6f/6f8 Chip Grade monoclonal antibody from Abcam. Western for Kaiso was done with Kaiso 12H monoclonal antibody from Santa Cruz Biotech.

Unless otherwise herein described, growth factors, such as recombinant human epidermal growth factor (“EGF”), were obtained commercially and was added to serum free media of DMEM and T-media with antibiotics.

Unless otherwise set forth herein, cell migration was assessed by the ability of the cells to move into an acellular area in a two-dimensional wound healing assay. At approximately 70-80% confluence, cells were detached and then re-plated at 1.0×10⁶ cells/well in 24-well culture plates in complete growth media DMEM and incubated for 24 hours at 37° C. in 5% CO₂. Cells were then washed with phosphate buffered saline solution (“PBS”), and the media were changed to DMEM containing 0.5% dialyzed FBS for 24 hours. A denuded area was generated in the middle of each well with a rubber policeman. The cells were then stimulated with EGF (10 nmol/L) in the presence or absence of siRNA Kaiso and then incubated for 24 hours. Images were taken at 0 and 24 hours, and the relative distance moved into the wounded area at the acellular front was determined. All treatments were normalized to the no treatment, which equals 1.

In experimental analysis of Kaiso, Applicants identified a consistent overall increase in expression of Kaiso as cancer progresses was identified. An increase in nuclear Kaiso was also identified in more advanced stages of cancer. Kaiso was also found to cause a delay in cell migration and invasion in vitro. Downregulation of Kaiso also rendered G1 arrest. These findings support that Kaiso can be used as a clinical indicator for cancer progression and that nuclear Kaiso expression may be used as an indicator that local and/or metastasis has occurred in patients.

Using immunohistochemistry, 303 prostate tumors were stained for Kaiso expression utilizing antibody to the F6 epitope. Initially, it was observed that Kaiso was weakly expressed in normal prostate tissue, however strong expression was observed in prostate tumors. Evaluation of staining was done utilizing a semiquantitative approach, previously shown to reproducibly identify cancer biomarkers in various tissue types. Briefly, each cell (pre-neoplasic or tumor) is evaluated separately for membranous, cytoplasmic and nuclear staining. Intensity of immunostaining for each tumor cells is classified with the percent of cells determined at each staining intensity from 0 to +4. Based on the utilized evaluation criteria, an overall increase of Kaiso expression in primary prostate tumors and lymph node metastasis was found when compared to normal samples. Table 1 shows the subcellular Kaiso expression with clincopathological features in prostate cancer. Further evaluation of individual samples determined that there was litter overlap between membrane, cytoplasmic, and nuclear expression, with nuclear expression associated with malignant and metastasis samples.

TABLE 1

| | | Kaiso expression | | | | |
|---|---|---|---|---|---|---|
| | (n = | Nuclear | | Cytomembrane | | |
| | 303) | N | N | C | C | P-value |
| Age (years, | | 68.13 | 0.7012 | 63.88 | 0.8750 | 0.0022 |
| mean S.D) | | | | | | |
| INVASION | | | | | | |
| T0-T1 | 14 | 1.286 | 0.1941 | 1.071 | 0.1469 | 0.1934 |
| T2-T4 | 57 | 1.316 | 0.08361 | 0.9737 | 0.065554 | 0.0008 |
| DIFFEREN- | | | | | | |
| TIATION | | | | | | |
| Well - | 55 | 1.218 | 0.06712 | 0.9182 | 0.06325 | 0.0015 |
| Moderate | | | | | | |
| Poor - Undif- | 103 | 1.320 | 0.05202 | 0.9272 | 0.04898 | <0.0001 |
| ferentiated | | | | | | |

To further evaluate Kaiso expression among racial groups, African American and Caucasian prostate cancer patient groups were compared. The results showed that nuclear Kaiso is differentially elevated in African American men compared to Caucasian men.

Since Kaiso positivity within the nuclear region in high grade prostate cancers was observed, tests were performed to analyze Kaiso expression and localization in commonly utilized prostate cancer cell culture models, DU-145 and PC-3, and a highly invasive and metastatic DU-145 subline, DU-145WT, that are genetically engineered to over-express EGFR. DU-145 cells showed mainly cytoplasmic Kaiso localization, while DU145WT and PC-3 showed cytoplasmic and nuclear localization. In addition, subcellular fractions of PC-3 cells and DU-145WT cells expressed Kaiso protein in the nuclear fraction compared to less aggressive DU-145 cells showing expression in only the cytoplasmic fraction. Lastly, increasing mRNA expression in DU-145WT compared to DU-145 cells was observed, however the most distinct observation was observed in PC-3 cells.

Since EGFR over-expressing DU-145WT cells express higher levels of Kaiso than the original DU-145 cells, experiments were further conducted to determine if EGFR signaling would have a direct effect on Kaiso localization and/or expression in prostate cancer cells. As an initial experiment, a dose dependent treatment of EGF consisting of 10, 50 and 100 ng/ml was performed. At least a two-fold increase in Kaiso mRNA and protein levels at the lowest level (10 ng/ml) utilizing real-time RT-PCR and immunoblot were observed, respectively. Using 10 ng/ml EGF treatment dosage, incremental Kaiso increases in DU-145 and PC-3 cells at the RNA and protein levels over a 24 hour period were observed. However, EGFR over-expression DU145WT cells showed 4-fold increases under identical treatment conditions.

Next, steps were taken to determine if EGFR signaling was involved in increasing Kaiso levels and/or cytoplasmic to nuclear trans-localization of Kaiso. All three cell lines were treated with EGF (10 ng/ml) over increasing time intervals of 30 minutes, 1 hour, and 24 hours. Visible nuclear accumulation of Kaiso was observed by immunofluorescence as early as 30 minutes after EGF treatment and continued to 24 hour. A key finding within DU-145 cells that lack nuclear Kaiso expression was the observation of a predominant Kaiso nuclear staining. These findings were further observed upon subcellular fractionation, where EGF treatment as early as 30 minutes resulted in a Kaiso nuclear localization shift. Interestingly, DU-145WT cells, which over-express EGFR, were found to have significant amounts of nuclear Kaiso expression that increases with EGF treatment. To determine the specificity of EGFR activity on Kaiso expression levels, an EGFR specific kinase inhibitor, PD153035, was also utilized in the presence or absence of EGF treatment. Experimental results show that 500 nM of PD153035 significantly reduced Kaiso expression levels, and PD153035 pre-treatment completely blocked the previously observed EGF induced increases in Kaiso levels in DU-145 and PC-3 cells. PD153035 treatment likewise resulted in a lack of nuclear Kaiso staining in DU-145 and PC-3 cells. Thus, these findings suggest that activation of the EGFR signaling cascade has a direct affect on Kaiso expression levels and localization.

Previous reports have observed that p120^{ctn} has a specific kinase activity for EGFR on tyrosine residue Y228. Additionally, p120^{ctn} is a known binding partner for Kaiso. Therefore, experiments were conducted determine whether this was apparent in prostate cancer cell lines as well. Given that p120^{ctn} has multiple tyrosine residues, including Y228, Y96, Y291, located within the regulator domain, the effect of EGF stimulation on each of these residues was studied. EGF treatment induced the specific increased expression of Y228, as opposed to Y96 and Y291 after 24 hours in both DU-145 and DU-145WT. It was observed by immunofluorescence that Y228 expression remained cytosolic after 24 hours of EGF treatment.

Given the specific role of p120^{ctn} during growth factor activation, and that cytoplasmic p120^{ctn} complexes with Kaiso have been observed, whether the presence of p120^{ctn} is required for EGFR induced kaiso nuclear shuttling was examined. When siRNA p120^{ctn} transfected DU-145 cells were compared to control transfected DU-145 cells, the siRNA p120^{ctn} transfected DU-145 cells failed to exhibit nuclear Kaiso expression over 24 hour period of EGF stimulation. Thus these findings suggest an important role for p120^{ctn} in Kaiso nuclear localization upon EGF stimulation.

Since Kaiso expression and localization is a result of the EGFR signaling cascade, experiments were also conducted to examine the effect of Kaiso on cell migration and invasion. To establish a functional role for Kaiso during prostate cancer cell migration and invasion, stable shRNA transfected DU-145 and PC-3 cells were established. shRNA Kaiso transfected cells show 80% decrease in Kaiso expression at RNA and protein levels. The migration rate of DU-145 or PC-3 cells transfected with Kaiso siRNA cells was reduced compared to the control transfectants. Further EGF stimulation failed to induce a mitogenic effect in Kaiso depleted cells, thus indicating that Kaiso is an important mediator of prostate cancer cell migration. For cancer cells to invade surrounding tissue, the cells must degrade the underlying basement membrane. To determine a role for Kaiso in prostate cancer cell invasion, the ability of DU-145 and PC-3 cells to invade the extracellular matrix was also evaluated. For this, PC-3 cells transfected with shKaiso or control shRNA vector were seeded onto a filter that was coated with Matrigel. Strikingly, suppression of endogenous Kaiso expression resulted in significant inhibition of prostate cancer cell invasion, resulting in a clear reduction in the ability of the cells to invade through Matrigel compared to the control shRNA vector only cells.

Loss of E-cadherin expression in human cancers is frequently associated with increased cell migration, cell invasion, and ultimately poor prognosis. The mode of E-cadherin downregulation has been reported to occur via growth factor induced decreases, or hypermethylation of E-cadherin promoter function. It has also been reported that activation of EGFR directly decreases E-cadherin expression in prostate cancer cell lines, and this is reversible with the addition pharmacological EGFR inhibitors. To determine the role of Kaiso in re-expression of E-cadherin, shKaiso cells were probed for E-cadherin. PC-3 cells showed increased E-cadherin at RNA and protein levels, this was associated with more epithelial morphology and cell aggregation. Collectively, this data suggest that nuclear Kaiso promotes EGFR induced cell migration and invasion through suppression of tumor suppressor E-cadherin.

Kasio expression in Human Prostate Tissue by IHC. Tissue samples including normal, malignant and lymph node metastases were stained and further analyzed. A score was given ranging from 0 to 3, o being no staining and 3 strong staining. Statistics were analyzed via students' t-test (Table 2).

TABLE 2

| | No. of | | | P-Value | | |
|---|---|---|---|---|---|---|
| | Cases | Mean | | Norm. vs | Mal. vs | P-Value |
| Tissue Type | (%) | Score | Age | Mal. | Met. | Age |
| Normal | 37 | 1.5< | 44.89 | | | |
| | | | | 0.0073 | | 0.1929 |
| Malignant | 82 | 1.5> | 47.19 | | | |
| | | | | | 0.0438 | 0.0438 |
| Lymph Node | 49 | 2≧ | 49.02 | | | |
| (LN) metastases | | | | | | |

The correlation of Kaiso expression with clincopathological features in prostate cancer is shown in Table 3.

TABLE 3

| | Kaiso expression | | | | |
|---|---|---|---|---|---|
| | Nuclear | | Cytomembrane | | |
| | (2-3)* | | (0-1)* | | P-value |
| Age (years, mean | 68.13 | 0.7012 | 63.88 | 0.750 | 0.0022 |
| S.D.) | | | | | |
| LYMPHATIC | | | | | |
| INVASION | | | | | |
| Tumor invades | 1.286 | 0.1941 | 1.071 | 0.1469 | 0.1934 |
| muscularis propria | | | | | |
| Tumor invades | 1.316 | 0.08361 | 0.9737 | 0.06554 | 0.0008 |
| through muscularis | | | | | |
| propria into subserosa | | | | | |
| or other organs | | | | | |
| DIFFERENTIATION | | | | | |
| Well | 1.455 | 0.1575 | 0.8182 | 0.1818 | 0.0155 |
| Moderate | 1.159 | 0.07229 | 0.9432 | 0.9432 | 0.0295 |
| Poor | 1.310 | 0.05260 | 0.9300 | 0.04925 | <0.0001 |
| Undifferentiated | 1.667 | 0.3333 | 0.8333 | 0.4410 | 0.2062 |

*Range of score used only for age category

The above-described experiments with respect to prostate cancer patients and cells lines further supports that subcellular localization of Kaiso correlates with increase in prostate cancer.

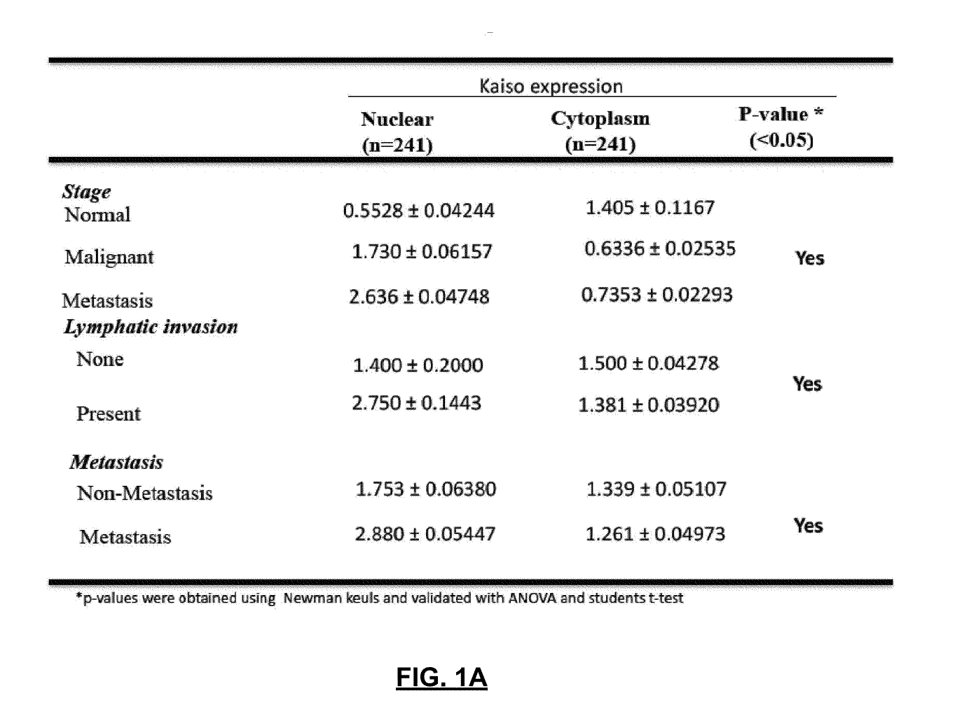

The expression of Kaiso in infiltrating ducal carcinomas (“Ides”) and lymph node metastasis was examined first by performing immunohistochemical (“IHC”) for infiltrating ductal (“IDC”) breast cancer progression TMA with anti-Kaiso 6F8 antibody. The tissues samples were stained and analyzed, and an overall score was given ranging from 0 to 3 (0 meaning no staining, and 3 meaning strong staining). The overall results of this IHC analysis for 241 total cases is reported in the table depicted by FIG. 1A, which data shows an overall weak expression of Kaiso in normal samples. However, there was a significant increase in Kaiso expression in malignant IDC tumors. Quantitatively, this amounted to significant increases in the mean expression levels with respect to normal versus malignant tissues and malignant versus metastasis tissues. However the most significant increase, as expected, was observed in the normal in comparison to metastasis tissues. Further, Applicants observed significant subcellular increases in the cytoplasmic and nuclear compartments, as shown in the table provided as FIG. 1B. Additionally, once the data was further split to consider cases of lymphatic invasion and metastasis, Applicants found correlations as shown in the table of FIG. 1C. Nuclear Kaiso expression in particular was significantly correlated with cancer stage and grade.

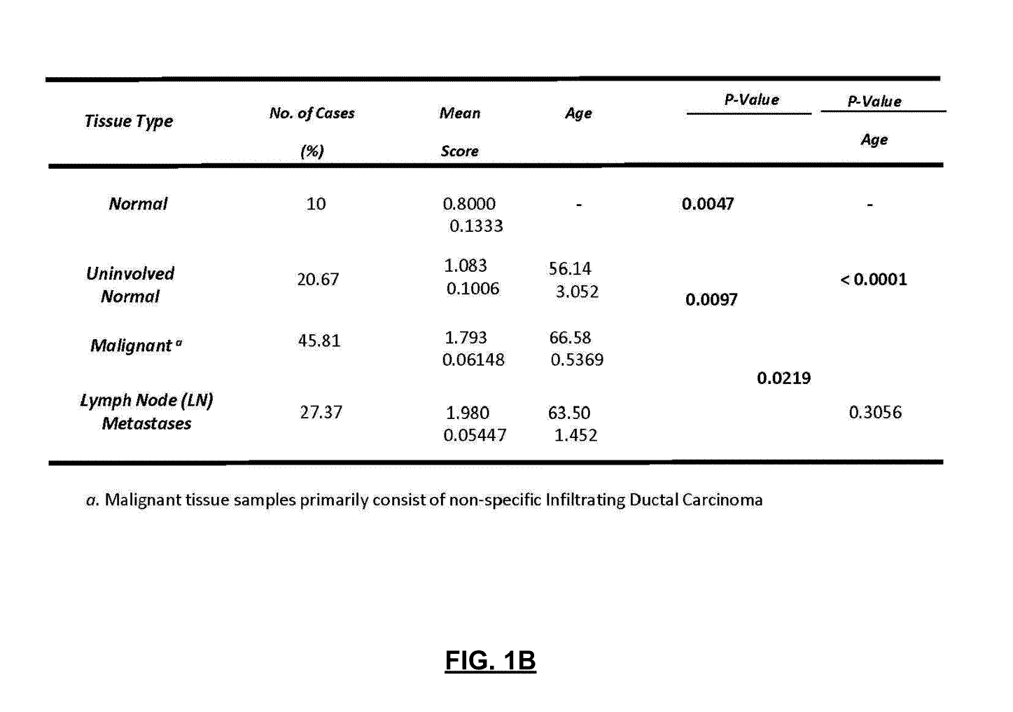

FIG. 2 is a scatter plot showing for the 241 total cases analyzed the overall, nuclear, cytoplasmic, and membranous Kaiso expression levels in normal and malignant breast tissue. Student's t-test confirmed that the overall and nuclear Kaiso expression levels of the malignant samples were higher than those of the normal samples, with p-values of 1.8×10⁻²⁴ and 2×10⁻²⁵, respectively.

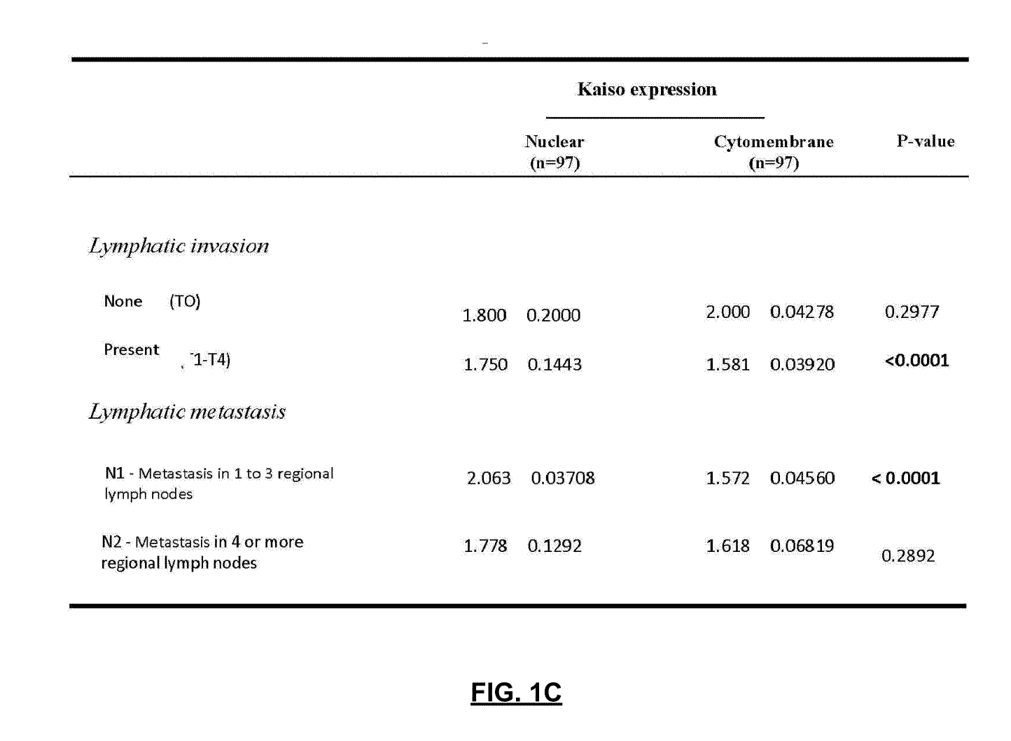

When Applicants sorted the data by racial origin, the data demonstrated that nuclear Kaiso expression was significantly over-expressed in malignant and metastasis patients from African American women. In particular, African American malignant samples were found to exhibit comparable expression levels to Caucasian metastasis samples, as shown by FIG. 3. Kaiso expression was uniformly observed throughout Applicants' analysis of tumor samples.

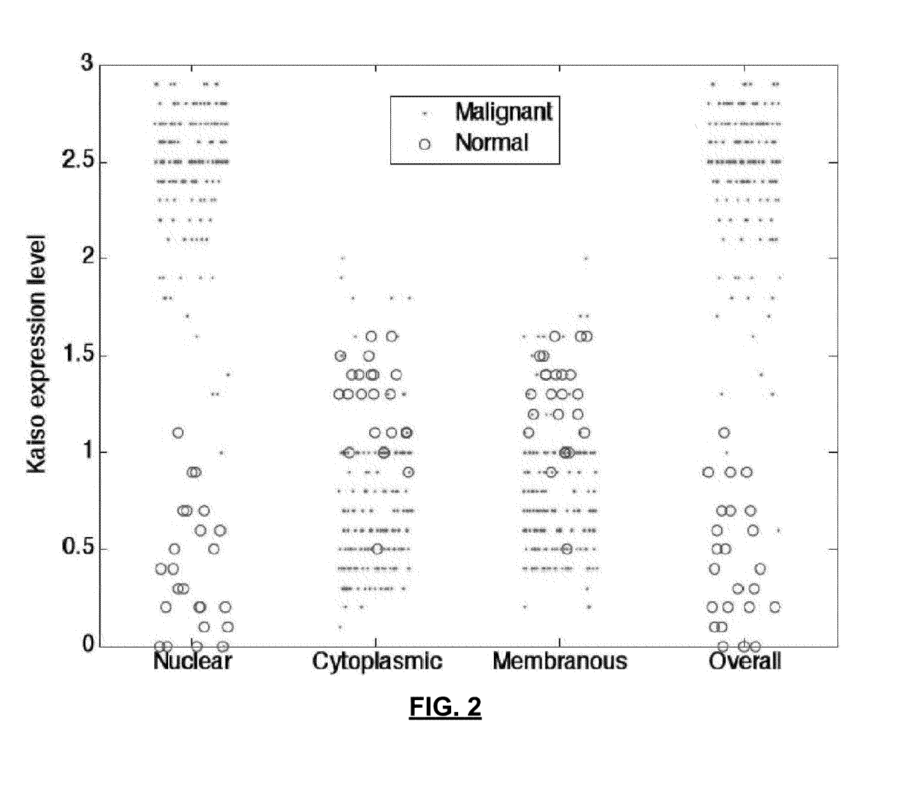

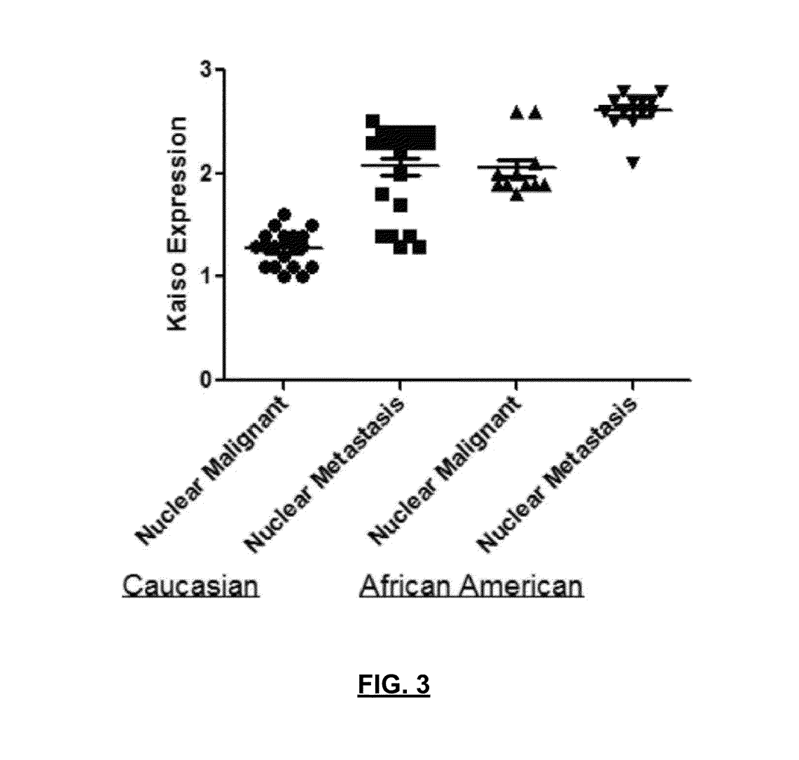

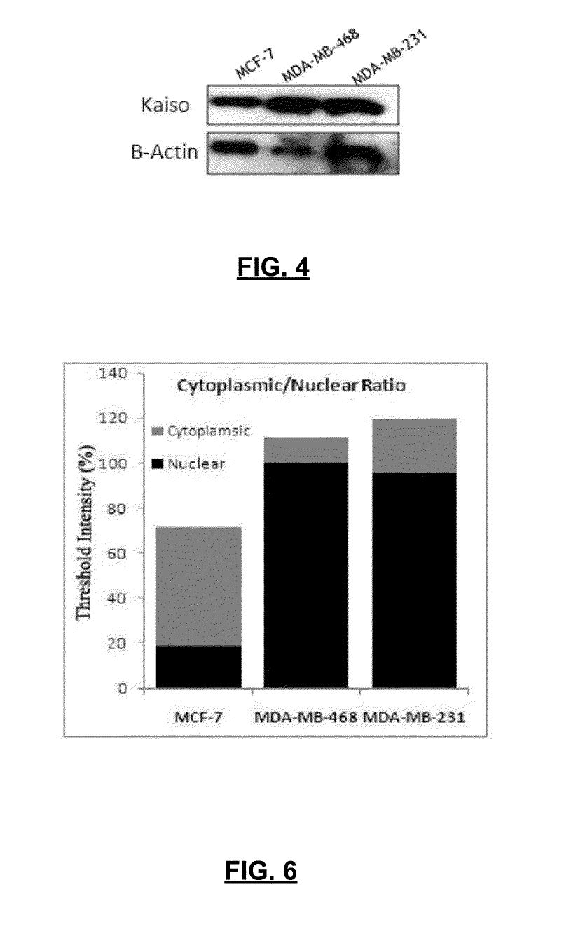

Since the experimental data demonstrated that Kaiso expression and localization clearly has a positive correlation with breast cancer progression, further experimental analyses were conducted to determine if this held true in commonly utilized cell culture models of breast cancer. The cell lines selected included MCF-7, an estrogen receptor positive human breast cancer cell line that can be tumorogenic in animal models when supplemented with estrogen, MDA-MB-468, an estrogen receptor-negative human breast cancer cell line, and MDA-MB-231, another estrogen receptor-negative human breast cancer cell line that is known to be relatively more aggressive in terms of tumorogenicity and metastasis incidence in animal models than MDA-MB-468. The results of these analyses are shown in FIG. 4 through FIG. 6. FIG. 4 is a comparison of gel images of PCR probing for mRNA Kaiso levels and protein for the three cell types, FIG. 5 is a grid of twelve color immunofluorescence photographs for the three cell lines, and FIG. 6 is a bar graph showing the relative threshold intensity identified for each of the cell lines. Briefly, as shown in these figures, immunofluorescence of the cells revealed that MCF-7 cells showed very little nuclear Kaiso, although strong cytoplasmic staining, as opposed to the metastatic MDA-MB-468 and MDA-MB-231 cells, which showed clear nuclear and cytoplasmic positivity. These findings suggest that Kaiso expression and subcellular localization is associated with breast cancer progression in patient sample and commonly utilized cell culture models for breast cancer.

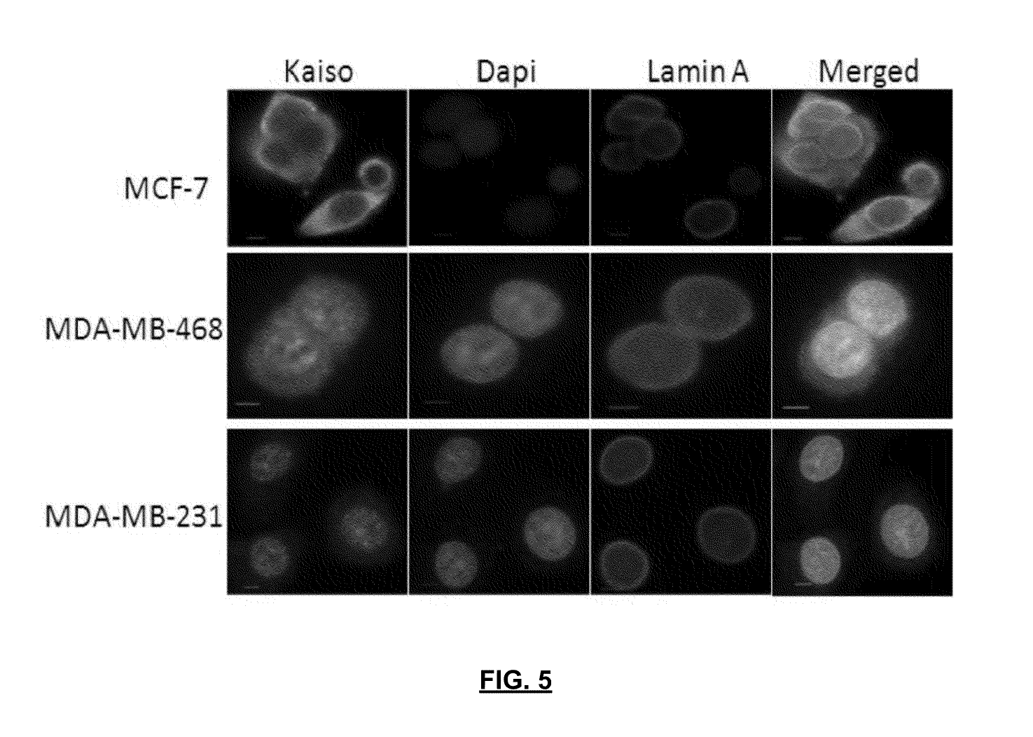

Since the data suggested a clinical correlation between Kaiso expression and breast cancer stage and grade, further experiments were conducted to investigate if Kaiso plays a functional role on the ability of breast cancer cells to display an increased ability to metastasize. To access the functional significance of Kaiso, the highly metastatic MDA-MB-231 cell was utilized in a series of experiments to measure levels of relative Kaiso expression as it correlates to cell migration. Two siRNA Kaiso constructs, identified as constructs 1 and 2, were utilized to determine efficiency of Kaiso depletion. The siRNA Kaiso construct 1 exhibited most significant decreases (70%, as shown in the bar graph of FIG. 7) and thus was further utilized in cell migration assays and invasion assays.

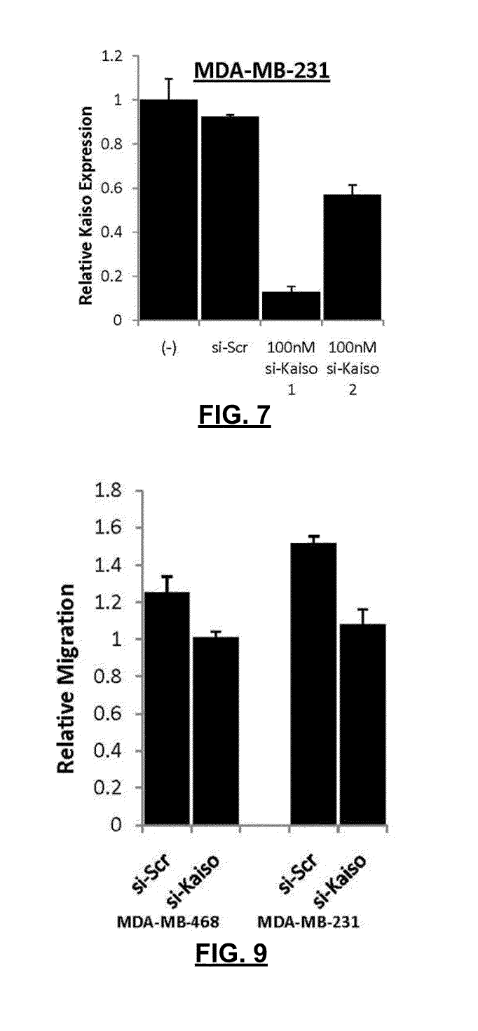

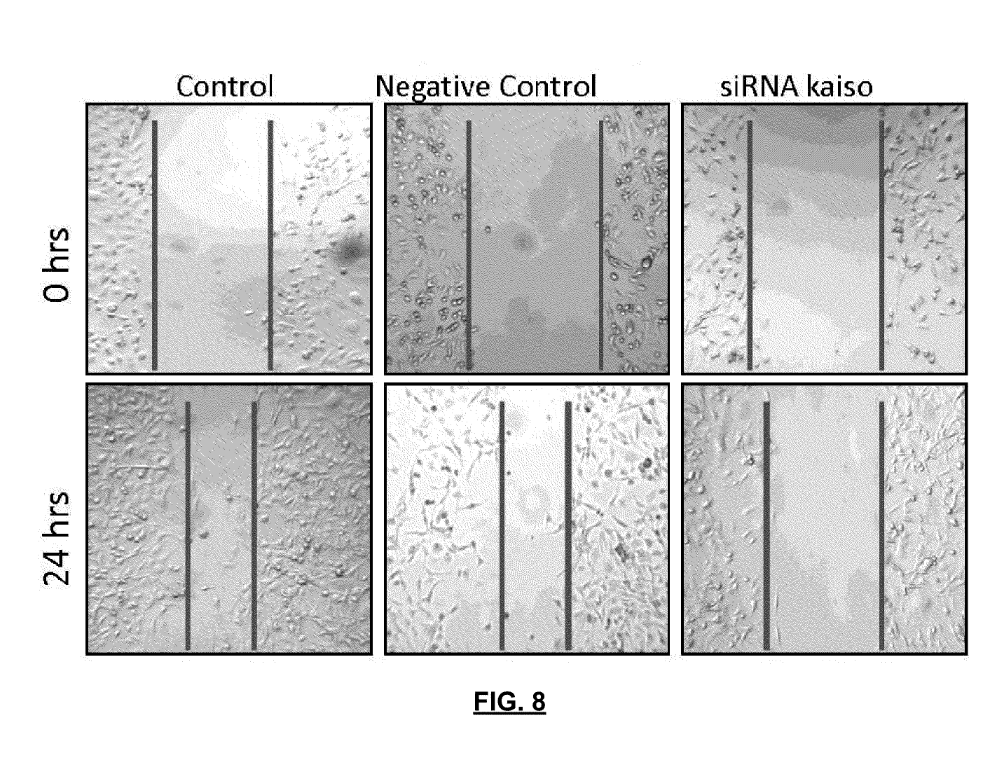

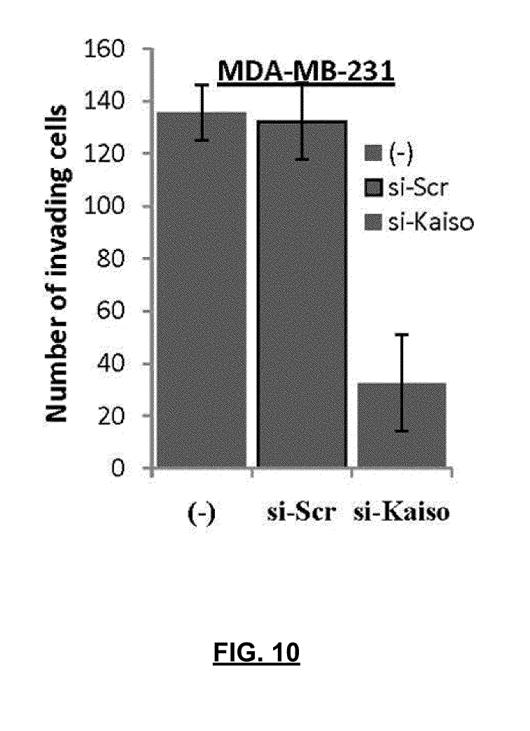

The cell migration and invasion assays were performed on both MDA-MB-231 and MDA-MB-468 cells. FIG. 8 comprises an array of six black and white photographs of representative cell cultures for the cell migration assay for MDA-MB-231 cells at zero and 24 hours. Applicants found that siRNA Kaiso treated cells showed a significant impairment in cell migration, specifically resulting in a delay in the distance of migrating cells compared to controls after 24 hours. The quantitative relative migration results for both cell lines is reported in the bar graph of FIG. 9. Similarly, Applicants found that siRNA Kaiso treated MDA-MB-231 cells showed a significant reduction in their invasive ability through a layer of Matrigel, as reported in the bar graph of FIG. 10. Lastly, siRNA Kaiso transfected MDA-MB-231 cells were found to exhibit an accumulation of cells in the G1 phase of cycle cell compared to controls.

Having described preferred embodiments of the invention, it will now become apparent to those of ordinary skill in the art that other embodiments incorporating these concepts may be used. Accordingly, it is submitted that that the invention should not be limited to the described embodiments but rather should be limited only by the spirit and scope of the appended claims.

Thus, although the invention has been described and illustrated with a certain degree of particularity, it is understood that the present disclosure has been made only by way of example, and that numerous changes in the combination and arrangement of steps, ingredients, or processes can be resorted to by those skilled in the art without departing from the spirit and scope of the invention, as will be claimed.

1. A method for the detection of breast or prostate cancer in a subject, said method comprising:
(a) obtaining a sample biopsy of breast or prostate tissue from a subject;
(b) determining an expression level of Kaiso in said sample biopsy; and
(c) comparing the expression level of Kaiso in said sample biopsy with an average expression level of Kaiso for a healthy control biopsy and an average expression level of Kaiso for a cancerous control biopsy, said cancerous control biopsy being of a known breast or prostate cancer stage;
wherein a determination that the level of expression of Kaiso in said sample biopsy is closer to the average level of expression of Kaiso in said cancerous control biopsy than to the average level of expression of Kaiso in said healthy control biopsy leads to a diagnosis of the subject as having breast or prostate cancer, and wherein a determination that the level of expression of Kaiso in said sample biopsy is greater than the average level of expression of Kaiso in said cancerous control biopsy leads to a diagnosis of the subject as having breast or prostate cancer of said stage.

2. The method according to claim 1, wherein said comparing step further comprises comparing the expression level of Kaiso in said sample biopsy with an average expression level of Kaiso for a second cancerous control biopsy, said second cancerous control biopsy being of a different breast or prostate cancer stage.

3. The method according to claim 2, further comprising determining a current stage of breast or prostate cancer for said subject based upon where said level of Kaiso in said sample biopsy falls in comparison to said average levels.

4. The method according to claim 2, wherein at least one of said stages comprises metastasis.

5. The method according to claim 2, wherein at least one of said stages is indicative of impending metastasis.

6. The method according to claim 2, wherein at least one of said stages is indicative of impending hormone independent breast or prostate cancer.

7. The method according to claim 2, wherein at least one of said stages is indicative of aggressive breast or prostate cancer.

8. The method according to claim 1, wherein said sample biopsy is a pathology sample taken from a cancerous tumor surgically removed from said subject.

9. The method according to claim 1, wherein said sample biopsy is taken from a lump of breast or prostate tissue.

10. The method according to claim 1, wherein said cancer is infiltrating duct breast cancer.

11. The method according to claim 1, wherein said subject is a patient undergoing breast or prostate cancer chemotherapy or radiotherapy.

12. The method according to claim 1, further comprising identifying said as being at risk of developing breast or prostate cancer if said sample biopsy level of Kaiso is determined to be higher by a statistically significant amount than said average level of Kaiso in said healthy control sample.

13. The method according to claim 1, wherein different average levels are used based upon racial or ethnic profiles of said subject.

14. A method for treating a subject diagnosed with breast or prostate cancer, said method comprising providing a therapy appropriate for the stage of breast or prostate cancer identified according to the detection method of claim 1.

15. A method for monitoring the stage of breast or prostate cancer in a subject, said method comprising:
(a) measuring the expression level of Kaiso in breast or prostate tissue of the subject;
(b) comparing the measured expression level of Kaiso with average expression levels of Kaiso for at least two breast or prostate cancer stages; and
(c) identifying the subject as having cancer of a particular stage depending upon said measured expression level falls relative to said average expression levels.

16. The method according to claim 15, wherein at least one of said stages is characterized by impending metastasis.

17. The method according to claim 15, wherein at least one of said stages is characterized by hormone independent breast or prostate cancer.

18. A method for treating a subject diagnosed with breast or prostate cancer, said method comprising providing a therapy appropriate for the stage of breast or prostate cancer identified according to the monitoring method of claim 15.

19. A method for detecting potential metastasis of breast or prostate cancer in a subject, said method comprising:
(a) measuring the expression level of Kaiso in breast or prostate tissue of the subject;
(b) comparing the measured expression level of Kaiso with average expression levels of Kaiso for at least two breast or prostate cancer stages, at least one of said stages being characterized by metastasis and at least another one of said stages being characterized by no metastasis; and
(c) identifying the subject as having increased risk for metastasis if said measured expression level falls relatively closer to said average expression level for said stages being characterized by metastasis.

20. A method for treating a subject diagnosed with breast or prostate cancer, said method comprising providing a therapy appropriate for the stage of breast or prostate cancer identified according to the detection method of claim 19.
